(19) United States
(12) Patent Application Publication
(10) Pub. No.: US 2016/0331426 A1
(43) Pub. Date: Nov. 17, 2016

(54) UNIVERSAL LOCKING DEVICE FOR STABILIZING AND FIXING BONES

(71) Applicant: KANGHUI MEDICAL INNOVATION CO., LTD., Changzhou (CN)

(72) Inventors: Kai TAO, Changzhou (CN); Hua YIN, Changzhou (CN); Kai JIANG, Changzhou (CN)

(21) Appl. No.: 15/220,363

(22) Filed: Jul. 26, 2016

Related U.S. Application Data
(63) Continuation-in-part of application No. PCT/CN2014/092025, filed on Nov. 24, 2014.

(30) Foreign Application Priority Data
Oct. 16, 2014 (CN) 201410547976.0

(51) Int. Cl. A61B 17/80; A61B 17/86; A61B 17/68

(52) U.S. Cl. CPC A61B 17/8057; A61B 17/683; A61B 17/8605

(57) ABSTRACT

A universal locking device for stabilizing and fixing bones, including: a steel plate including a threaded hole, and a locking screw including a threaded rod and a screw head. The screw head includes a universal groove. The screw head includes a periphery having an external thread. The external thread corresponds to the threaded hole of the steel plate.

### CROSS-REFERENCE TO RELATED APPLICATIONS

This application is a continuation-in-part of International Patent Application No. PCT/CN2014/092025 with an international filing date of Nov. 24, 2014, designating the United States, now pending, and further claims priority benefits to Chinese Patent Application No. 201410547976.0 filed Oct. 16, 2014. The contents of all of the aforementioned applications, including any intervening amendments thereto, are incorporated herein by reference.

### BACKGROUND OF THE INVENTION

1. Field of the Invention

The invention relates to orthopedic surgery, and in particular, to a universal locking device for stabilizing and fixing bones and joints of the body.

2. Description of the Related Art

Conventional locking mechanisms for stabilizing and fixing bones include a locking screw and a steel plate. The locking angle formed by the locking screw and the steel plate are preset and fixed. However, there are different kinds of bone injuries, and individual bones differ in shapes. As a result, conventional devices for stabilizing and fixing bones do not achieve a satisfactory locking effect.

### SUMMARY OF THE INVENTION

In view of the above-described problems, it is one objective of the invention to provide a universal locking device for stabilizing and fixing bones which has wide range of implant direction and good locking effect. The angle between the locking screw and the steel plate of the universal locking mechanism is adjustable, so as to provide a proper implant direction in accordance with the fracture characteristics.

To achieve the above objective, in accordance with one embodiment of the invention, there is provided a universal locking device for stabilizing and fixing bones, comprising a steel plate and a locking screw. The steel plate comprises a threaded hole. The threaded hole of the steel plate is a tapered threaded hole.

The locking screw comprises a threaded rod and a screw head. The screw head comprises a universal groove. The universal groove is a plurality of longitudinal grooves on the screw head. The periphery of the screw head is provided with an external thread to which corresponds to the threaded hole of the steel plate.

The external thread on the periphery of the screw head is a tapered thread.

Working principle of the universal locking device for stabilizing and fixing bones is that: in use, an optimal implant direction is chosen in accordance with the fracture characteristics of bone. The steel plate with the thread hole is disposed on the fracture site, and a universal drill guide and a drill are employed to drill a hole on the bone, and the hole corresponds to the implant direction. As the locking screw comprises the universal groove, the threads are capable of being staggered, so that the locking screw is fixed in the threaded hole of the steel plate in an appropriate angel via a screwdriver. Then, the direction of the screw into the fracture site is the optimal direction for operation.

Advantages of the universal locking device for stabilizing and fixing bones according to embodiments of the invention are that: the locking mechanism features a proper design and a simple structure. The arrangement of the universal groove enables the angle between the locking screw and the steel plate of the universal locking mechanism is adjustable, which ensures the locking device for stabilizing and fixing bones has wide range of implant direction and good locking effect.

### BRIEF DESCRIPTION OF THE DRAWINGS

FIG. 1 is a schematic diagram of a locking screw of a universal locking device for stabilizing and fixing bones in accordance with one embodiment of the invention; and

FIG. 2 is a fixation diagram of a universal locking device for stabilizing and fixing bones in a fracture site in accordance with one embodiment of the invention.

In the drawings, the following reference numbers are used: 1. Threaded rod; 2. Screw head; 3. Universal groove; 4. Steel plate; 5. Threaded hole; 6. Screwdriver; 7. Bone; 8. External thread.

### DETAILED DESCRIPTION OF THE EMBODIMENTS

For further illustrating the invention, experiments detailing a universal locking device for stabilizing and fixing bones are described below. It should be noted that the following examples are intended to describe and not to limit the invention.

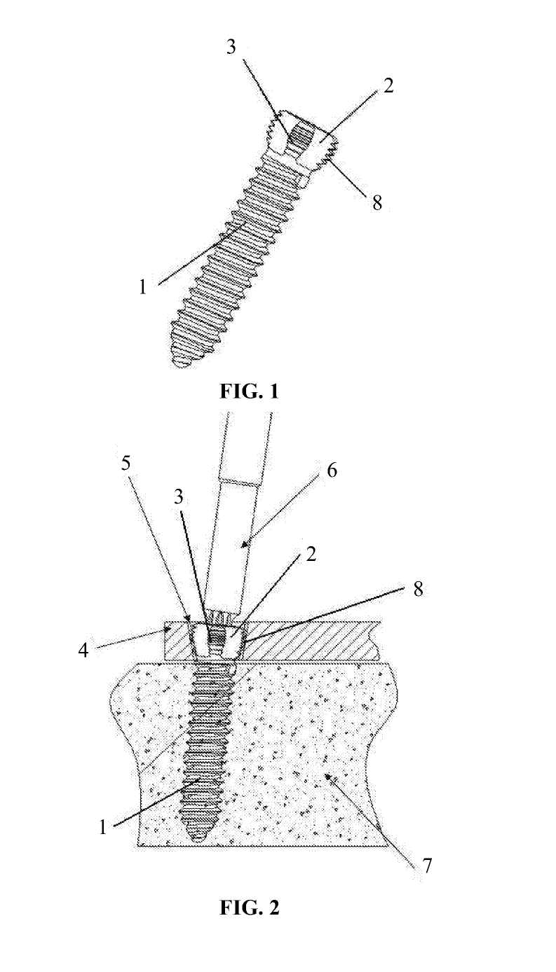

As shown in FIGS. 1-2, a universal locking device for stabilizing and fixing bones comprises a steel plate 4 and a locking screw. The steel plate 4 comprises a threaded hole 5. The threaded hole 5 on the steel plate 4 is a tapered threaded hole.

The locking screw comprises a threaded rod 1 and a screw head 2. The screw head 2 comprises a universal groove 3. The universal groove 3 is a plurality of longitudinal grooves on the screw head. The periphery of the screw head 2 is provided with an external thread 8 which corresponds to the threaded hole of the steel plate 4.

The external thread on the periphery of the screw head 2 is a tapered thread.

In operation, an optimal implant direction is decided in accordance with the fracture characteristics of bone. The steel plate 4 with the thread hole 5 is disposed on the fracture site, and a drill is employed to drill a hole on the bone 7 along the optimal implant direction. The threaded rod 1 passes through the threaded hole 5 and is fixed in the bone 7 via a screwdriver 6. Then the screwdriver 6 continues to screw, and the universal groove 3 of the screw head 2 and the thread of the threaded hole 5 on the steel plate are staggered. There is an included angle between the axis of rotation of the locking screw and the axis of rotation of the threaded hole. In the course of screwing the locking screw, due to the axial tension, the external thread of the screw head is forced into the staggered threaded hole. As a result, the screw head 2 and the threaded hole 5 on the steel plate are tightly screwed up at a certain angel, thus achieving the optimal implant of the locking screw.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A locking device for stabilizing and fixing bones, comprising:
a) a steel plate, the steel plate comprising a threaded hole; and
b) a locking screw, the locking screw comprising a threaded rod and a screw head;
wherein
the screw head comprises a universal groove;
the screw head comprises a periphery comprising an external thread; and
the external thread corresponds to the threaded hole of the steel plate.

2. The locking device of claim 1, wherein the threaded hole of the steel plate is a tapered threaded hole.

3. The locking device of claim 1, wherein the universal groove comprises a plurality of longitudinal grooves on the screw head.

4. The locking device of claim 1, wherein the external thread on the periphery of the screw head is a tapered thread.
